(19) United States
(12) Patent Application Publication
(10) Pub. No.: US 2010/0239502 A1
(43) Pub. Date: Sep. 23, 2010

(54) IN VIVO EXPRESSION ANALYSIS USING ULTRASOUND-INDUCED TRANSFECTION OF REPORTER CONSTRUCTS

(75) Inventors: Evan Edward Santo, Amsterdam (NL); Nevenka Dimitrova, Pelham Manor, NY (US); CHIEN Ting Chien, Tarrytown, NY (US)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/668,551

(22) PCT Filed: Jun. 26, 2008
(86) PCT No.: PCT/IB08/52572

§ 371 Date: Jun. 9, 2010

Related U.S. Application Data
(60) Provisional application No. 60/949,027, filed on Jul. 11, 2007.

(51) Int. Cl. A61K 49/00; C12N 15/85; C12Q 1/68

(52) U.S. Cl. 424/9.6; 435/320.1; 435/6

(57) ABSTRACT

The invention features compositions and methods for in vivo expression analysis. The data presented herein demonstrates that ultrasound-enhanced delivery and/or expression of a composition for expression analysis comprising microbubbles vectors as well as a genetic payload, comprising a “always-on” promoter, a “reference” reporter gene, a “query” promoter and an “answer” reporter gene, enables in vivo analysis of gene expression both without requiring prior preparation (especially genetic modification) of the test subject (animal or patient) and without causing long term or systemic effects on the subject. Such an invention can be used, for example, to query the epigenotypic or phenotypic response of the individual subject to a foreign effector substance such as a pyrogen, pharmaceutical compound, pharmaceutical lead compound, an allergen, an autoimmunogene, a toxin, a polyclonal antibody, a monoclonal antibody, an antigen, a lipid, a carbohydrate, a peptide, a protein, a protein-complex, an amino acid, a fatty acid, a nucleotide, DNA, RNA, PNA, siRNA and micro RNA.

### FIELD OF THE INVENTION

The present invention relates to a composition for expression analysis in mammals. The invention is in the field of biology and chemistry, more in particular in the field of diagnostics. The invention relates particularly to in vivo expression analysis.

### BACKGROUND OF THE INVENTION

The study of gene regulation is a burgeoning discipline of the biological sciences. It has been understood for quite some time that the transcription of genes into (coding and non-coding) RNAs is in most cases controlled by non-coding regulatory regions that are immediately 5-prime (or “upstream”) of the coding region. These non-coding sequences of DNA contain sequence patterns that determines the conditions (when, where and under what external stimulations) in which a gene is expressed during the lifetime of an organism. These non-coding sequences are bound by transcription factors (which are usually termed enhancers or repressors depending on their function) which in turn recruit epigenetic regulators and other proteins that form larger complexes and exert control over expression of the target transcript. Understanding the mechanisms by which genes are regulated is fundamental to understanding the progression of disease and everything from the most fundamental to the most complex biological processes.

Over the years, many ways have been devised to study gene expression at both the regulatory and expression levels. On an individual level, gene expression is usually measured quite reliably using a technique known as reverse transcriptase polymerase chain reaction (RT-PCR). In order to gauge expression analysis of thousands of genes in parallel, techniques such as microarrays, serial analysis of gene expression (SAGE) and massively parallel signature sequencing (MPSS) have been employed. To infer the patterns of gene expression many approaches to statistical analysis have been developed.

The drawback is that all of these methods require physical isolation and destruction of either the tissue or cells that contain the RNAs of interest. This makes the study of gene expression costly and labor intensive. Studying gene expression over time is especially complicated because of the need to take many samples and synchronize the gene expression of all the cells in the sample. Most importantly when the sample is treated with various reagents, one can not avoid “shocking” the cells which distorts the expression pattern. It would therefore be desirable to have at hand an in vivo expression analysis system. However, bringing an expression system into an organ or even a tissue in such a manner that no side-mediated expression occurs is also a problem that has to do with the problem of transfecting said vector in a directed and effective manner.

Thus, there is also a need for a practical and effective vector delivery method combined with such an expression analysis system. The primary problem of vector injection by conventional needle-syringe methods is that the vector material must be injected in large quantities into the target site because of the inefficiencies of attempting to diffuse vector material into the cell's nucleus and the problem that enzyme systems immediately move to destroy the injected vector nucleic acid molecules. For example, therapeutic injection technology using a needle-syringe has progressed relatively slowly.

For example, cationic liposomes have been widely used for gene transfer into endothelial cells in vivo (Brigham, K. B. et al. (1989) Am. J. Med. Sci. 298, 278-281; Hofland, H. E. J. et al. (1997), Pharm. Res. 14, 742-749; Liu, F. et al. (1997), Gene Therapy 4, 517-523; Mahato, R. I. et al. (1998), Hum. Gene. Ther. 9, 2083-2099; Rolland, A. P. (1998), Critical Reviews in Therapeutic Drug Carrier Systems 15,1 43-198). The utility of current cationic liposome-based systems for targeting tumor endothelium is limited due to lack of target cell specificity and low in vivo gene transfer efficiency (Lesoon-Wood, L. A. et al. (1995), Hum. Gene. Ther. 6, 395-405; Anwer et al. (Human gene Therapy, submitted)).

Modification of liposome surface by covalent conjugation of monoclonal antibodies or other targeting moieties (e.g., specific peptides and lipids) has been proposed to improve tumor-specific gene delivery (Boulikas, T. (1996), Int. J. Oncol. 9, 941-954; Kong, H. L., and Crystal, R. G. (1998), J. Natl. Cancer Inst. 90, 273-286; Pietersz, G. A. and McKenzie, I. F. C. (1992), Immunol. Rev. 129, 57-80; Thorpe, P. E. and Derbyshire, E. J. (1997), J. Cont. Release 48, 277-288; Kircheis, R. et al. (1997), Gene Therapy 4, 409-418).

Mechanical methods such as electroporation and jet injection have also been described as useful external means to enhance gene transfer in target tissue (Gallo, S. A. et al. (1997), Biophys. J. 72, 2805-2811).

Ultrasound-mediated delivery has the potential as a powerful new method for enhancing and targeting administration of therapeutic compounds into and across cells and tissues. Ultrasound-enhanced delivery to cells has been demonstrated in vitro by uptake of extracellular fluid, drugs, and DNA into cells (Liu, J. et al. (1998), Pharm. Res. 15, 918-924; Mitragotri, et al. (1996), Pharm. Res. 13, 411-420; Wyber, J. A. et al. (1997), Pharm. Res. 14, 750-756; Tata, D. B., et al. (1997), Biochem. Biophys. Res. Commun. 234, 64-67).

### SUMMARY OF THE INVENTION

The invention features compositions and methods for in vivo expression analysis. The data presented herein demonstrates that ultrasound-delivery of a composition for expression analysis comprising microbubbles as well as a mammalian expression vector system enables in vivo analysis of gene expression both without a foreign effector substance as well as upon provision of a foreign effector substance such as a pyrogen, pharmaceutical compound, pharmaceutical lead compound, an allergen, an autoimmunogene, a toxin, a polyclonal antibody, a monoclonal antibody, an antigen, a lipid, a carbohydrate, a peptide, a protein, a protein-complex, an amino acid, a fatty acid, a nucleotide, DNA, RNA, PNA, siRNA and micro RNA.

The invention relates to a composition for expression analysis of a specific query “X” comprising (a) a vector comprising of microbubbles either encapsulating or associating with the genetic payload, (b) a genetic payload comprising (i) a fluorescent reporter gene which is under the control of a promoter which will ensure constitutive expression in vivo, (ii) a second fluorescent reporter gene which is under the control of a promoter that is not constitutively expressed, (v) a promoter for the first fluorescent reporter gene, and (vi) a promoter for the second fluorescent reporter gene which is activated conditional on the in vivo status of “X”, (c) an ultrasonic device which interacts with the microbubble vector in order to release the payload and/or enhance its uptake by the cells and/or enhance its expression in the cells and (d) a readout device or method which quantifies the expression levels of both the first and second reporter genes and infer from the results the relevant answer to query “X”.

Since the interrogation subjects the animal or patient to a minimal level of trauma, the expression analysis can be repeated as soon as the genetic payload disappears from the target tissues or cells (typically a few days). If the ultrasonic device can trigger the microbubbles vector locally, query “X” can be repeated almost immediately in comparable tissues or cells previously not interrogated. Either way, temporal repeatability is a unique benefit of the invention.

Since expression of the genetic payload can be activated with the ultrasonic device in most soft tissues of most strains and species of animals, expression analysis can be performed without restricting to specially genetically-engineered animals. Virtually universal applicability to most animals is another unique benefit of the invention.

As used herein, the term “microbubble” refers to emulsified stabilized bubbles with mean size smaller than 10 μm (1-3 μm being most typical). Special gases, typically high molecular weight inert gases such as C₄F₁₀ and SF₆, are encapsulated in these bubbles to increase in vivo stability to the order of minutes to hours. Bubble shells are made of lipids, polysaccharides, albumins or other polymers. Specific manufacturing steps and augmentations prevent aggregation (clumping) and coalescence (merging) of bubbles. Additionally, the bubbles are made non-immunogenic by attaching polyethylene glycol (PEG) or other biologically “stealth” molecules to the shell. Several diagnostic products are currently in use for ultrasound imaging in cardiac, radiological and oncological settings. Attaching molecular ligands to these bubbles for molecular imaging is currently in research phase. Encapsulating, attaching or associating therapeutic molecules, including conventional drugs and genetic materials, are also being studied as novel approaches to deliver local interventions while minimizing systemic side-effects.

As used herein, the term “expression vector system” refers to a construct, made up of genetic material (i.e. nucleic acids). It includes genetic elements arranged such that an inserted coding sequence can be transcribed in eukaryotic cells. Also, while the plasmid may include a sequence from a viral nucleic acid, such viral sequence preferably does not cause the incorporation of the plasmid into a viral particle and the plasmid is therefore a non-viral vector. Preferably, such a vector is a closed circular DNA molecule. The expression vector as used herein refers to a construction comprised of genetic material designed for direct transformation of a targeted cell. It contains preferably contiguous fragments of DNA or RNA, positionally and sequentially oriented with other necessary elements such that the nucleic acid can be transcribed and when necessary translated in the transfected cells.

The term “transfection facilitating agent” as used herein refers to an agent that forms a complex with the vector described above. This molecular complex is associated with the vector molecule in a covalent or a non-covalent manner. The transfection facilitating agent should be capable of transporting nucleic acid molecules in a stable state and of releasing the bound nucleic acid molecules into the cellular interior. In addition, the transfection facilitating agent may prevent lysosomal degradation of the nucleic acid molecules by endosomal lysis. Furthermore, the transfection facilitating agent may allow for efficient transport of the nucleic acid molecule through the cytoplasm of the cell to the nuclear membrane and into the nucleus and provide protection.

In one embodiment transfection facilitating agents are non-condensing polymers, oils and surfactants. Non-condensing polymers have been found to be particularly suitable for injection into the site of desired expression such as in intra-tumoral administration. These may be suitable for use when the expression vector requires prolonged localization. In some instances it may be useful to have for example, a sustained release of the expression vector according to the invention. Thus, some of the following compounds may be useful in the context of the present invention:

Polyvinylpyrrolidones; polyvinylalcohols; propylene glycols; polyethylene glycols; polyvinylacetates; poloxamers (Pluronics) (block copolymers of propylene oxide and ethylene oxide, relative amounts of the two subunits may vary in different poloxamers); poloxamines (Tetronics); ethylene vinyl acetates; celluloses, including salts of carboxymethylcelluloses, methylcelluloses, hydroxypropyl-celluloses, hydroxypropylmethylcelluloses; salts of hyaluronates; salts of alginates; heteroploysaccharides (pectins); phosphatidylcholines (lecithins); miglyols; polylactic acid; polyhydroxybutyric acid.

In another embodiment, cationic condensing agents such as cationic lipids, peptides, or lipopetides, or for example, dextrans, chitosans, dendrimers, polyethyleneiminie (PEI), or polylysine, may be associated with the vector according to the invention and may facilitate transfection and conjunction with the ultrasonic target microbubble destruction.

Some of the compounds mentioned above may be used as, and are considered, protective, interactive, non-condensing compounds (PINC) and others are sustained release compounds, while some may be used in either manner under the respectively appropriate conditions.

The PINC enhances the delivery of the nucleic acid molecule i.e. the vector according to the invention to mammalian cells in vivo and preferably the nucleic acid molecule, i.e. the vector includes a coding sequence as will be outlined in more detail below for a promoter for a gene product to be expressed in said cell.

The expression vector according to the invention may also be complexed with a liposome formed from the one or more cationic lipids. Preferably the cationic lipid is DOTMA and the neutral co-lipid is cholesterol (chol). DOTMA is 1,2-di-O-ocatadecenyl-3-trimethylammonium propane, which his described and discussed in Eppstein et al., U.S. Pat. No. 4,897,355, issued Jan. 30, 1990, which is incorporated herein by reference. However, other lipids and lipid combinations may be used in other embodiments. A variety of such lipids are described in Gao & Huang, 1995, Gene Therapy 2:710-722, which is hereby incorporated by reference.

As the charge ratio of the cationic lipid and the DNA is also a significant factor, in preferred embodiments the DNA and the cationic lipid are present in such amounts that the negative and positive charge ratio is about 1:3. Thus, preferably the charge ratio for the composition is between about 1:1 and 1:10, more preferably between about 1:2 and 1:5.

The term “cationic lipid” refers to a lipid which has a net positive charge at physiological pH, and preferably carries no negative charges at such pH. An example of such a lipid is DOTMA.

The term “sonoporation device”, as used herein relates to an apparatus that is capable of causing or causes uptake of nucleic acid molecules, i.e. the vector according to the invention into the cells of an organism by ultrasound means. The cell membrane may thus be destabilized and result in the formation of passage ways or pores in the cell membrane. The type of sonoporation device is not considered a limiting aspect of the present invention. The primary importance of a sonoporation device is, in fact, the capability of the device to deliver formulated nucleic acid molecules, i.e. the vector according to the invention into the cells of an organism.

The term “organism” as used herein refers to common usage by one of ordinary skill in the art. The organism can include; micro-organisms, such as yeast or bacteria, plants, birds, reptiles, fish or mammals. The organism can be a companion animal or a domestic animal. Preferably the organism is a mammal. Preferred mammals include mouse, rat, chimpanzee, dog and other mammals used in clinical research.

The use of the expression vector according to the invention in human beings will depend on whether or not its diagnostic or therapeutic application is possible.

The term “fluorescent reporter gene” refers to a gene which is able to express a protein that when excited with the necessary wave length is able to fluoresce or produce light.

For the purpose of the present invention a fluorescent reporter gene may be any gene which produces a protein when excited with an appropriate wave length results in emission of a light signal that may be detected. In a preferred embodiment the emission spectrum from the fluorescent protein according to the invention is between 445-660 nm, between 550-660 nm and most preferably between 550-660 nm.

Further, in a preferred embodiment the fluorescent protein according to the invention has a quantum yield of higher than 0.10, preferably higher than 0.20, more preferably higher than 0.40, most preferably higher than 0.50 and very most preferably higher than 0.60. A quantum yield as well as an emission spectrum between 550-660 nm has been shown in the present invention to be of great advantage due to better in vivo results.

The terms “mammalian origin of replication”, “bacterial origin of replication”, “promoter” as well as “ubiquitously” expressed herein refer to common usage by one of ordinary skill in the art.

### BRIEF DESCRIPTION OF THE DRAWINGS

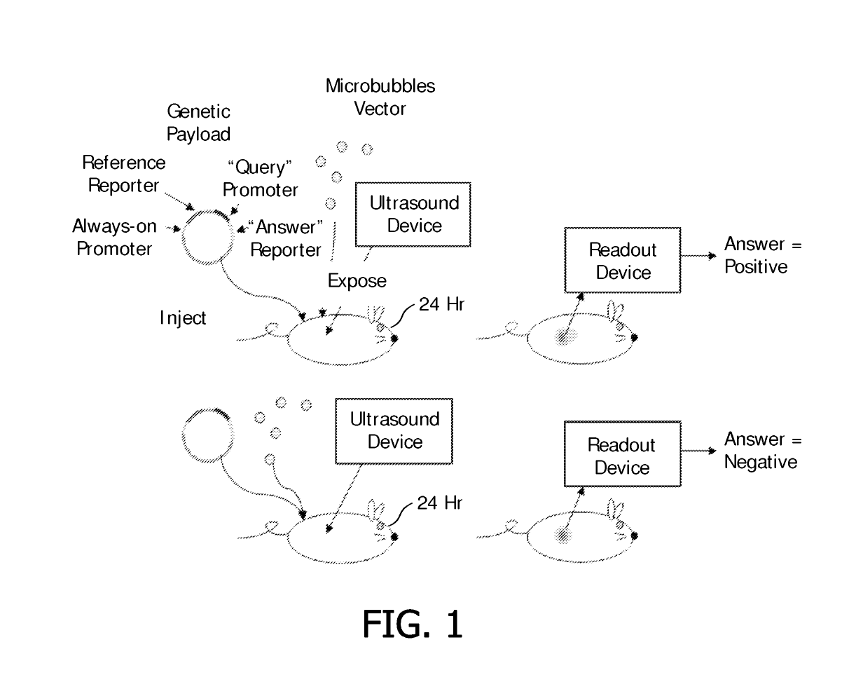

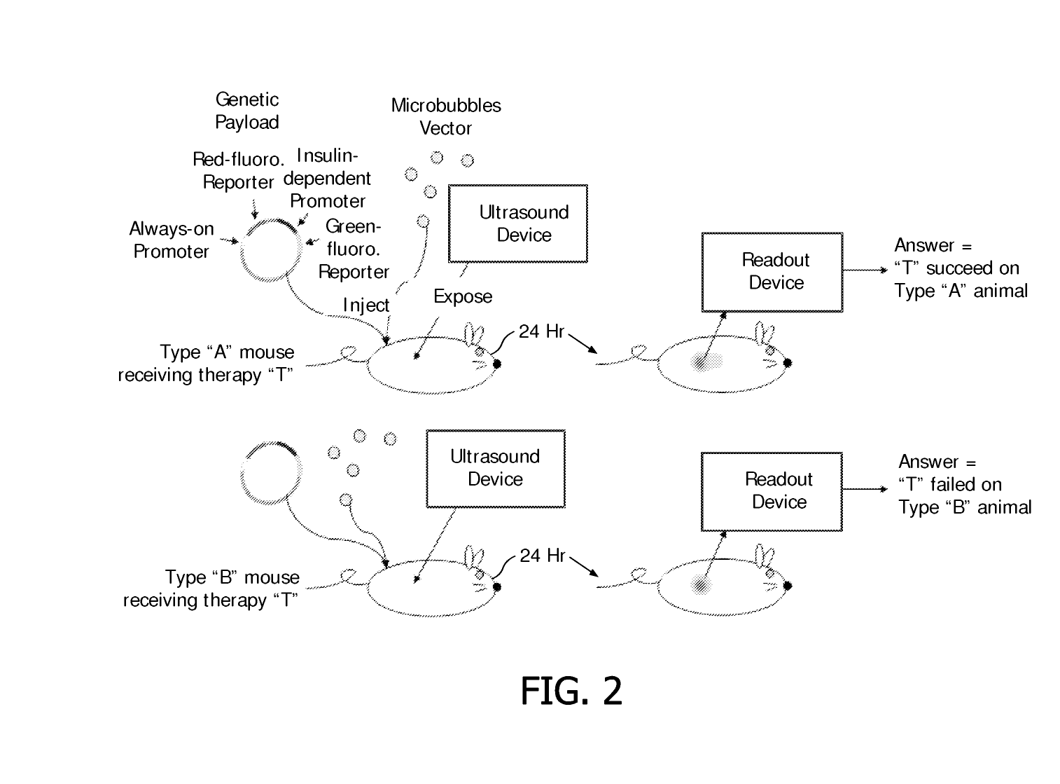

The drawings show both the principle of the invention (FIG. 1) as well as a preferred embodiment thereof (FIG. 2).

### DETAILED DESCRIPTION OF EMBODIMENTS

The inventors have astonishingly found that by using a composition for expression analysis comprising, (a) microbubbles, (b) a mammalian expression vector system comprising, (i) a first fluorescent reporter gene which is under the control of a promoter which will ensure constitutive expression in vivo, (ii) a second fluorescent reporter gene which is under the control of a promoter that is not constitutively expressed, (iii) a mammalian origin of replication, (iv) a bacterial origin of replication, (v) a promoter for the first fluorescent reporter gene, and (vi) a promoter for the second fluorescent reporter gene which is activatable and ubiquitously expressed it is possible to perform in vivo expression analysis. In a preferred embodiment the mammalian vector system comprises one or more further fluorescent reporter genes, which are under the control of a promoter that is not constitutively expressed.

In a preferred embodiment the promoter of said second or further fluorescent reporter gene stems from the organism into which the expression vector system according to the invention is to be shuttled.

Further, it is preferred that the one or more further fluorescent reporter genes are under the control of a promoter that is not constitutively expressed.

For example, the promoter may stem from hormone genes, cytokine genes, an insuline gene, an interleukin gene, a somatropine gene, an erythropoietin gene, an interferon gene, in particular erythropoietin-α, interferon-γ, interferon-α as well as erythropoietin-α from granulocyte macrophage-stimulating factor (GM-CSF). Interferon, plasmino gene activator, a glucocerebrosidase gene, calcitonin gene, a growth factor gene, genes involved in hepatitis A/B/C/D/E as well as any other gene involved in, e.g. a human disease. For example, the promoter may stem from a gene that is involved in cancer development. Such a gene may be, e.g. a cell cycle control gene, tumor suppressor gene, a gene involved in apoptosis or the like.

The present inventors have found that by bringing the present expression vector system into the tissue, at the site of the expression of the second or further promoter on the vector system it is for the first time possible to address, e.g. drug development questions in vivo with respect to gene expression.

In a preferred embodiment the first fluorescent reporter gene is selected from the group of genes encoding, green fluorescent protein, blue fluorescent protein, cyan fluorescent protein, yellow fluorescent protein, red fluorescent protein, destabilized green fluorescent protein. The second or further fluorescent reporter gene is selected from the group of genes encoding, green fluorescent protein, blue fluorescent protein, cyan fluorescent protein, yellow fluorescent protein, red fluorescent protein and destabilized green fluorescent protein.

In a preferred embodiment the promoter for the first fluorescent reporter gene is selected from the group of cytomagelovirus promoter (CMV), CMV-IE, HIV-1 long terminal repeat (LTR) encoding the transcriptional promoter LTR, SV40 IE, HSV tk, β-actin, human globin α, human globin β, and human globin γ promoter.

In a preferred embodiment the microbubble medium is comprised of a medium selected from the group of free gas bubbles, stabilized gas bubbles, colloidal suspension, emulsions, and aqueous solution.

In a preferred embodiment the microbubble medium is a colloidal suspension comprising dodecafluorpentane.

In a particularly preferred embodiment the microbubble medium is an aqueous solution comprised of sonicated albumin.

As outlined above, the green fluorescent protein according to the invention may be any of green fluorescent protein, blue fluorescent protein, cyan fluorescent protein, yellow fluorescent protein, orange and red fluorescent proteins. Other fluorescent proteins are also possible. However, certain fluorescent proteins are preferred.

In case a green fluorescent protein is chosen, it is preferentially selected from the group of EGFG, AcGFP, TurboGFP, Emerald, Azani Green and ZsGreen.

In case a blue fluorescent protein is chosen, it is preferentially selected from the group of EBFP, Sapphire and T-Sapphire.

Choosing cyan fluorescent protein one would select a protein from the group of ECFP, mCFP, Cerulean, CyPet, amCyanl, Midori-Ishi Cyan and mTFP1 (Teal).

One may also choose a yellow fluorescent protein in this case one may select a protein from the group of EYFP, Topaz, Venus, mCitrine, Ypet, PhiYFP, ZsYello1 and mBanana.

When choosing an orange or red fluorescent protein preferentially the protein is selected from Kusabira Orange, mOrange, dTomato, dTomato-Tandem, DsRed, DsREd2, DsRed-Expresss (T1), DSRed-Monomer, mTangerine, mStrawberry, AsRed2, mRFP1, Jred, mCherry, HcRed1, mRaspberry, HcRed-Tandem, mPlum and AQ143.

A selection of fluorescent protein is shown in Table 1.

Fluorescent Protein Properties

| | Excitation | Emission | Molar | | | Relative |
|---|---|---|---|---|---|---|
| Protein | Maximum | Maximum | Extinction | Quantum | in vivo | Brightness |
| (Acronym) | (nm) | (nm) | Coefficient | Yield | Structure | (% of EGFP) |
| GFP (wt) | 395/475 | 509 | 21,000 | 0.77 | Monomer* | 48 |

Green Fluorescent Proteins

| | | | | | | |
|---|---|---|---|---|---|---|
| EGFP | 484 | 507 | 56,000 | 0.60 | Monomer* | 100 |
| AcGFP | 480 | 505 | 50,000 | 0.55 | Monomer* | 82 |
| TurboGFP | 482 | 502 | 70,000 | 0.53 | Monomer* | 110 |
| Emerald | 487 | 509 | 57,500 | 0.68 | Monomer* | 116 |
| Azami Green | 492 | 505 | 55,000 | 0.74 | Monomer | 121 |
| ZsGreen | 493 | 505 | 43,000 | 0.91 | Tetramer | 117 |

Blue Fluorescent Proteins

| | | | | | | |
|---|---|---|---|---|---|---|
| EBFP | 383 | 445 | 29,000 | 0.31 | Monomer* | 27 |
| Sapphire | 399 | 511 | 29,000 | 0.64 | Monomer* | 55 |
| T-Sapphire | 399 | 511 | 44,000 | 0.60 | Monomer* | 79 |

Cyan Fluorescent Proteins

| | | | | | | |
|---|---|---|---|---|---|---|
| ECFP | 439 | 476 | 32,500 | 0.40 | Monomer* | 39 |
| mCFP | 433 | 475 | 32,500 | 0.40 | Monomer | 39 |
| Cerulean | 433 | 475 | 43,000 | 0.62 | Monomer* | 79 |
| CyPet | 435 | 477 | 35,000 | 0.51 | Monomer* | 53 |
| AmCyan1 | 458 | 489 | 44,000 | 0.24 | Tetramer | 31 |
| Midoriishi Cyan | 472 | 495 | 27,300 | 0.90 | Dimer | 73 |

Yellow Fluorescent Proteins

| | | | | | | |
|---|---|---|---|---|---|---|
| EYFP | 514 | 527 | 83,400 | 0.61 | Monomer* | 151 |
| Topaz | 514 | 527 | 94,500 | 0.60 | Monomer* | 169 |
| Venus | 515 | 528 | 92,200 | 0.57 | Monomer* | 156 |
| mCitrine | 516 | 529 | 77,000 | 0.76 | Monomer | 174 |
| YPet | 517 | 530 | 104,000 | 0.77 | Monomer* | 238 |
| PhiYFP | 525 | 537 | 124,000 | 0.39 | Monomer* | 144 |
| ZsYellow1 | 529 | 539 | 20,200 | 0.42 | Tetramer | 25 |
| mBanana | 540 | 553 | 6,000 | 0.7 | Monomer | 13 |

Orange and Red Fluorescent Proteins

| | | | | | | |
|---|---|---|---|---|---|---|
| Kusabira Orange | 548 | 559 | 51,600 | 0.60 | Monomer | 92 |
| mOrange | 548 | 562 | 71,000 | 0.69 | Monomer | 146 |
| dTomato | 554 | 581 | 69,000 | 0.69 | Dimer | 142 |
| dTomato-Tandem | 554 | 581 | 138,000 | 0.69 | Monomer | 283 |
| DsRed | 558 | 583 | 75,000 | 0.79 | Tetramer | 176 |
| DsRed2 | 563 | 582 | 43,800 | 0.55 | Tetramer | 72 |
| DsRed-Express (T1) | 555 | 584 | 38,000 | 0.51 | Tetramer | 58 |
| DsRed-Monomer | 556 | 586 | 35,000 | 0.10 | Monomer | 10 |
| mTangerine | 568 | 585 | 38,000 | 0.30 | Monomer | 34 |
| mStrawberry | 574 | 596 | 90,000 | 0.29 | Monomer | 78 |
| AsRed2 | 576 | 592 | 56,200 | 0.05 | Tetramer | 8 |
| mRFP1 | 584 | 607 | 50,000 | 0.25 | Monomer | 37 |
| JRed | 584 | 610 | 44,000 | 0.20 | Dimer | 26 |
| mCherry | 587 | 610 | 72,000 | 0.22 | Monomer | 47 |
| HcRed1 | 588 | 618 | 20,000 | 0.015 | Dimer | 1 |
| mRaspberry | 598 | 625 | 86,000 | 0.15 | Monomer | 38 |
| HcRed-Tandem | 590 | 637 | 160,000 | 0.04 | Monomer | 19 |
| mPlum | 590 | 649 | 41,000 | 0.10 | Monomer | 12 |
| AQ143 | 595 | 655 | 90,000 | 0.04 | Tetramer | 11 |

In a preferred embodiment the composition according to the invention comprises two or more genes encoding the two or more fluorescent proteins and these proteins exhibit an emission spectrum which differs by at least 20 nm, 30 nm, 40 nm, 50 nm, 60 nm, 70 nm or preferably 80 nm or more. Ideally, when using two or more fluorescent proteins, a selection of proteins is performed in such a way that the emission spectra do not overlap. Thus, in a preferred embodiment when using for example three fluorescent proteins, the emission spectra of each of the three proteins is separated by at least 20 nm, preferentially 30 nm or more. Obviously when using four or more fluorescent proteins, due to the number of proteins available with a limited amount of emission spectra it is only possible to have separations of possible 20 nm, 30 nm or 40 nm. However, when using only two fluorescent proteins it may be possible to have proteins separated by an emission spectrum, of e.g. 80 nm.

At the same time it is important that the two or more genes encoding the two or more fluorescent proteins encode fluorescent proteins which exhibit a relative brightness in percent of Enhanced Green Fluorescent Protein (EGFP) of 60%, 80%, 100%, 120%, 160%, 180% or preferably more. Thus, the inventors have found that while it is important that the emission spectra of the two or more fluorescent proteins are separated, it is likewise important that the selected fluorescent proteins exhibit a relative brightness that enables in vivo use.

In one aspect of the invention, the invention concerns a composition wherein the composition additionally comprises a substance selected from the group of a transfection facilitating agent.

In a preferred embodiment the composition according to the invention has microbubbles which have encapsulated the mammalian expression vector system. In any case it is necessary to have encapsulated the mammalian expression vector system prior to its application to the organism.

The invention also concerns a method for in vivo expression analysis comprising the steps of (a) provision of a composition according to the invention, (b) application of the composition according to (a) to an organism, to a tissue or organ of interest, (c) ultrasonic target microbubble destruction of the composition of step (b) at the site of the tissue or organ of interest with a sonoporation device, (d) excitation of at least one of the two or more proteins, encoded by the two or more reporter genes, by application of a light source, wherein the light source emits a light with a wavelength corresponding to the excitation range or preferably excitation maximum of the two or more fluorescent proteins, (e) detection of expression of the first fluorescent reporter gene, (f) detection of expression of the second or further fluorescent reporter gene, and optionally (g) repeating steps (d) to (f).

In a preferred embodiment of the method for in vivo expression analysis according to the invention the method additionally comprises the steps of (a) applying a potential effector substance to the organisms prior to a first repetition of steps (d) to (f), wherein said effector substance is thought to possibly induce the transcription of a second or further reporter genes. In this embodiment of the invention it is for the first time possible to analyze the expression reaction of an organism in vivo upon application of a potential effector substance. It is clear that such a substance could for example be a pharmaceutical compound or a pharmaceutical lead compound. Thus, in this application of the invention it is possible to test, for example, toxic side effects of a given drug candidate. One would for example, have an expression vector system which has a promoter of an immunological gene of interest which is known to produce a toxic side effect such as some interleukins or cytokines which when expressed lead to severe immunological side effects. Then, one would apply as an effector substance the drug candidate and test in vivo whether or not a second or further reporter gene, which is under the control of the interleukin or cytokine promoter is induced and consequently leads to light emission. Thus, for the first time the invention allows in vivo expression analysis in cases where toxic side effects of pharmaceutical lead compounds are to be analyzed.

However, in a preferred embodiment numerous different effector substances may be chosen. Thus, these may be selected, e.g. from the group of a pyrogen, a pharmaceutical compound, a pharmaceutical lead compound, an allergen, an autoimmunogen, a toxin, a polyclonal antibody, a monoclonal antibody, an antigen, a lipid, a carbohydrate, a peptide, a protein, a protein complex, an amino acid, a fatty acid, a nucleotide, a DNA, RNA, PNA, siRNA and microRNA.

In a further embodiment the invention relates to the use of the composition according to the invention for diagnosis and/or therapy. The invention also concerns a kit comprising the composition according to the invention.

In one embodiment the invention relates to a method for in-vivo expression analysis comprising the steps of, provision of a composition according to the invention, application of the composition according to (a) to an organism, to a tissue or organ of interest, ultrasonic target microbubble destruction of the composition of step (b) at the site of the tissue or organ of interest with a sonoporation device, excitation of at least one of the two or more proteins, encoded by the two or more reporter genes, by application of a light source, wherein the light source emits a light with a wavelength corresponding to the excitation range or preferably excitation maximum of the two or more fluorescent proteins, detection of expression of the first fluorescent reporter gene, detection of expression of the second or further fluorescent reporter gene wherein the promoter for the second fluorescent reporter gene is the promoter of the phosphoenolpyruvat carboxy kinase gene.

### EXAMPLES

### Type II Diabetes Insulin Resistance

Type II diabetes insulin resistance causes glucose levels in the blood to be abnormally high. This occurs because the insulin signaling pathway is damaged; functional insulin signaling would normally result in stabilization of blood glucose levels. In type II diabetes increased insulin levels that result from feeding fail to trigger the insulin signaling pathway. It is also possible that insulin production itself is decreased. In either case, this failure allows cells of the liver and adipose tissue to continue converting lipids and glycogen stores to glucose, despite the fact that glucose is abundant from the from the food recently digested. This is how abnormally high blood glucose levels are achieved. Therefore, it would be of interest to identify compounds that reduce the conversion of glycogen and lipids to glucose in the liver and adipose tissue.

Many pathways play a role in the regulation of cell metabolism. A prominent pathway directly lined to insulin signaling is the PI3K/Akt pathway. This pathway directly regulates the activity of a family of transcription factors known as FOXO. When insulin levels are low in healthy individuals, the PI3K pathway is inactivated. This inactivation allows the FOXO family of transcription factors to localize to the nucleus and regulate various target genes involved in metabolism and other processes. Many of these target genes promote the breakdown of carbohydrates and lipids to glucose. One of these target genes, phophoenolpyruvate carboxykinase (PEPCK), is up-regulated primarily by FOXO family member FOXO1. Although other genes may be chosen, this link to the insulin signaling cascade via regulation by FOXO1 and the direct involvement of this gene in metabolism makes PEPCK a particularly useful “reporter” of functional insulin pathway activity. When insulin signaling activity is high, PEPCK expression is repressed, when insulin signaling activity is low, PEPCK expression is activated.

### Reporter Construction

Any suitable mammalian expression vector can be used as the “backbone” for reporter construction. At the very least, this vector must replicate as well as a selectable bacterial marker. Examples of suitable backbones would be commercially available. One such as phRG-B or pCAT®3-Basic available from Promega.

Using standard genetic engineering techniques, the chosen backbone modified to contain four essential elements. A) The fluorescent reporter to be used as the control. This is the one that will constitutively express and act as a visual marker of successful transfection. B) the fluorescent reporter that will be under the control of the promoter or regulatory region under study. C) the promoter that will drive the expression of the constitutively expressed fluorescent reporter. Cloned up stream of the constitutively expressed promoter. D) The promoter or regulatory region under study, cloned up stream of the fluorescent reporter chosen as the experimental output.

Ideally the control fluorescent reporter should be a stable, long-lived variant of the fluorescent reporters that has excitation and emission spectra considerably different from the reporter used for the study. An example would be the commercially available DsRed Monomer from Clontech (available in vector pDsRed-Monomer). The choice of the “study” reporter may depend on many factors aside from spectral properties. Since the primary application here is the measurement of gene expression, a destabilized fluorescent protein is preferred. This will increase the turnover rate considerably, which will allow for more sensitive detection of regulatory shifts that affect expression. However, the turnover rate cannot be so high that the detectable levels of fluorescence do not accumulate—detectable being defined by the sensitivity of the imaging apparatus and transfection efficiency achieved by ultrasound. One appropriate protein is the destabilized GFP described in U.S. Pat. No. 6,306,600 and commercially available from Clontech in the vector pZsGreen1-DR. The choice of the promoter for the constitutively expressed reporter is also important and will control the level of expression achieved by the control reporter. Ideally, this promoter should be ubiquitously expressed in the tissues of the organism under study and generally be expressed at low and stable levels. Expression here should be just enough to provide the reliable detection of successfully transfected cells. For strong expression, the ubiquitously expressed CMV promoter could be used. For more moderate expression, perhaps the promoter beta-actin or some other gene with very stable, moderate and ubiquitous expression. The “study” promoter or regulatory region of interest, in this particular embodiment it is the promoter of PEPCK.

The plasmid is then purified from the bacterial host, coupled to ultrasound “bubbles”, site-injected into the target tissue and transfected by application of the appropriate ultrasound frequency.

### Ultrasound Transfection

Ultrasound-enhanced gene transfection involves four components: (1) microbubble preparation, (2) equipment to combine the microbubble with the aforementioned DNA construct, (3) equipment (which may be as simple as a syringe) to inject the DNA microbubble combination into the animal, (4) an ultrasound device that activates the transfection.

Simultaneous imaging, particularly ultrasound imaging, has been shown to provide precise placement of the transfection zone and guide the setting of ultrasound parameters for transfection. The key technology challenge is to achieve high transfection rate in the target zone and minimalize side effects such as killing or injuring cells and non-specific transfection. Since both transfection rate and cell killing increase with ultrasound exposure, careful setting of intensity is critical for success. Some techniques, such as dynamic imaging of bubble destruction, may provide feedback for precise control of ultrasound intensity.

Selective transfection of a specific type of cells within an organ may be desired. In some cases, this can be achieved if a ligand to the specific cell type is available. A quantity of the ligand, typically an antibody, can be conjugated to the surface of the microbubbles in a preferred embodiment. When sufficient time is given the microbubbles selectively bind to the desired cell type, enhancing transfection rate to the relevant population of cells.

### Interaction of Transfected Genes

After transfection and allowance of the appropriate time of control, e.g. dsRed expression and fluorescence to occur, imaging may be performed to assess the efficiency of transfection and localization. If this is satisfactory, evaluation of a compound, gene therapeutic technique or any other treatment can be carried out. In this particular case, multiple mice (e.g. both disease model and control mice) have been ultrasound transfected in the aforementioned manner with the PEPCK reporter construct. Each mouse receives a different compound or combination of compounds and treatments. For each of these mice, the fluorescence intensity produced by the destabilized form of GFP is monitored by fluorescence imaging. In this study, compounds or treatments of interest result in the quenching of GFP fluorescence in either the liver, adipose tissue or both. The decrease in fluorescence over time is correlated with PEPCK promoter activity, indicating that a compound, treatment or combination of factors has been identified that may attenuate uncontrolled metabolism of lipids and glycogen to glucose in vivo in the tissue of interest. The compounds in fact may not necessarily impact the PI3K/Akt pathway or FOXO functionality. They may impact parallel pathways or other cellular components that impact the regulation of the PEPCK promoter specifically. More globally, the identified factors may influence the processes of transcription or translation, which would also produce an effect on PEPCK reporter activity.

### Fluorescence Imaging

GFP is both chemically and photochemically a very stable and resilient fluorophore. Using fluorescent proteins for the readout of gene expression is not straightforward due to endogenous autofluorescence. To solve the problem of autofluorescence it is important to have no possible sources thereof. There are a variety of substances that may cause autofluorescence such as flavin, NADH, lipofuscin, collagen, lignin and others. In general we use filters to pick out areas of the spectrum where GFP can be excited and its fluorescence transmitted with efficiency which is greater than the autofluorescence of molecules as mentioned above.

The excitation and emission spectra of enhanced blue, cyan, green, yellow and DsRed proteins are widely diverse. The bright green fluorescence emission from GFP (Imax=508-515 nm) is readily induced by illumination of the molecule with blue light (Imax=470 nm).

One of the standard techniques known in the art for such as photobleaching or photo conversion is used to remove the auto fluorescence.

In the whole process of imaging and determining the best fluorophore we envision a use of a decision support system which takes into consideration the ultrasound and imaging devices as well as the reporter constructs in order to provide easier guidance and monitoring during the whole research setup.

### FIGURE CAPTIONS

FIG. 1 shows the principle of the invention.

FIG. 2 To determine if experimental therapy “T” against type-1 diabetes is effective on both types “A” and “B” animals, the “query” promoter is an insulin dependent promoter.

1. Composition for expression analysis comprising,
a. microbubbles,
b. a mammalian expression vector system comprising,
i. a first fluorescent reporter gene which is under the control of a promoter which will ensure constitutive expression in vivo,
ii. a second fluorescent reporter gene which is under the control of a promoter that is not constitutively expressed,
iii. a mammalian origin of replication,
iv. a bacterial origin of replication,
v. a promoter for the first fluorescent reporter gene, and
vi. a promoter for the second fluorescent reporter gene which is activatable and ubiquitously expressed.

2. Composition according to claim 1, wherein the mammalian vector system comprises one or more further fluorescent reporter genes which, are under the control of promoter that is not constitutively expressed.

3. Composition according to claim 1, wherein
i. the first fluorescent reporter gene is selected from the group of genes encoding, green fluorescent protein, blue fluorescent protein, cyan fluorescent protein, yellow fluorescent protein, red fluorescent protein, destabilized green fluorescent protein,
ii. the second or further fluorescent reporter gene is selected from the group of genes encoding, green fluorescent protein, blue fluorescent protein, cyan fluorescent protein, yellow fluorescent protein, red fluorescent protein, destabilized green fluorescent protein,
iii. the promoter for the first fluorescent reporter gene is selected from the group of cytomegalovirus promoter (CMV), CMV-IE, HIV-1 long terminal repeat (LTR) encoding the transcriptional promoter LTR, SV40 IE, HSV tk, β-actin, human globin α, human globin β and human globin γ promoter, and
iv. the microbubble medium is comprised of a medium selected from the group consisting of free gas bubbles, stabilized gas bubbles, colloidal suspensions, emulsions, and aqueous solutions.

4. Composition according to claim 3, wherein the microbubble medium is a colloidal suspension comprising dodecafluoropentane.

5. Composition according to claim 3, wherein the microbubble medium is an aqueous solution comprised of sonicated albumin.

6. Composition according to claim 3, wherein
a. the green fluorescent protein is selected from the group of EGFG, AcGFP, TurboGFP, Emerald, Azani Green and ZsGreen,
b. the blue fluorescent protein is selected from the group of EBFP, Sapphire and T-Sapphire,
c. the cyan fluorescent protein is selected from the group of ECFP, mCFP, Cerulean, CyPet, AmCyan1, Midori-Ishi Cyan and mTFPI (Teal),
d. the yellow fluorescent protein is selected from the group of EYFP, Topaz, Venus, mCitrine, Ypet, PhiYFP, ZsYellow1 and mBanana,
e. the orange and red fluorescent proteins are selected from Kusabira Orange, mOrange, dTomato, dTomato-Tandem, DsRed, DsRed2, DsRed-Express (T1), DSRed-Monomer, mTangerine, mStrawberry, AsRed2, mRFP1, Jred, mCherry, HcRed1, mRaspberry, HcRed-Tandem, mPlum and AQ143.

7. Composition according to claims 1 to 6, wherein the two or more genes encoding the two or more fluorescent proteins encode fluorescent proteins which exhibit emission spectra which differs by at least 20 nm, 30 nm, 40 nm, 50 nm, 60 nm, 70 nm or preferably 80 nm or more.

8. Composition according to claim 1, wherein the two or more genes encoding the two or more fluorescent proteins encode fluorescent proteins which exhibit a relative brightness in % of Enhanced Green Fluorescent Protein (EGFP) of 60%, 80%, 100%, 120%, 160%, 180% or preferably more.

9. Composition according to claim 1, wherein the composition additionally comprises a substance selected from the group of, a transfection facilitating agent.

10. Composition according to claim 1, wherein the mammalian expression vector system is encapsulated by the microbubbles.

11. Method for in-vivo expression analysis comprising the steps of,
a. provision of a composition according to claim 1,
b. application of the composition according to (a) to an organism, to a tissue or organ of interest,
c. ultrasonic target microbubble destruction of the composition of step (b) at the site of the tissue or organ of interest with a sonoporation device,
d. excitation of at least one of the two or more proteins, encoded by the two or more reporter genes, by application of a light source, wherein the light source emits a light with a wavelength corresponding to the excitation range or preferably excitation maximum of the two or more fluorescent proteins,
e. detection of expression of the first fluorescent reporter gene,
f. detection of expression of the second or further fluorescent reporter gene, and optionally
g. repeating steps (d) to (f).

12. Method for in-vivo expression analysis according to claim 11, additionally comprising the steps of applying a potential effector substance to the organism prior to a first repetition of steps (d) to (f).

13. Method for in-vivo expression analysis according to claim 12, wherein
the effector substance is selected from the group of,
a pyrogen, a pharmaceutical compound, a pharmaceutical lead compound, an allergen, an autoimmunogen, a toxin, a polyclonal antibody, a monoclonal antibody, an antigen, a lipid, a carbohydrate, a peptide, a protein, a protein complex, an amino acid, a fatty acid, a nucleotide, DNA, RNA, PNA, siRNA and microRNA.

14. Use of a composition according to claim 1 for diagnosis and/or therapy.

15. Kit comprising a composition according to claim 1.

16. Method for in-vivo expression analysis comprising the steps of,
a. provision of a composition according to claim 1
b. application of the composition according to (a) to an organism, to a tissue or organ of interest,
c. ultrasonic target microbubble destruction of the composition of step (b) at the site of the tissue or organ of interest with a sonoporation device,
d. excitation of at least one of the two or more proteins, encoded by the two or more reporter genes, by application of a light source, wherein the light source emits a light with a wavelength corresponding to the excitation range or preferably excitation maximum of the two or more fluorescent proteins,
e. detection of expression of the first fluorescent reporter gene,
f. detection of expression of the second or further fluorescent reporter gene, wherein the promoter is for the second fluorescent reporter gene is the promoter of the phosphoenolpyruvat carboxy kinase gene.
